(19) United States
(12) Plant Patent Application Publication
(10) Pub. No.: US 2003/0005503 P1
(43) Pub. Date: Jan. 2, 2003

(54) Chrysanthemum plant named 'Gedi One Mis'

(76) Inventor: Dirk Pieters, Staden (BE)

Correspondence Address: KNOBBE MARTENS OLSON & BEAR LLP, 2040 MAIN STREET, FOURTEENTH FLOOR, IRVINE, CA, 92614, US

(21) Appl. No.: 09/873,905

(22) Filed: Jun. 4, 2001

(51) Int. Cl. A01H 5/00

(52) U.S. Cl. PLT/293

(57) ABSTRACT

A new and distinct Chrysanthemum plant variety characterized by a decorative type inflorescence, consistent flowering response to short days, blooming consistently after 49 days of short day length, free branching habit, and compact growth habit.

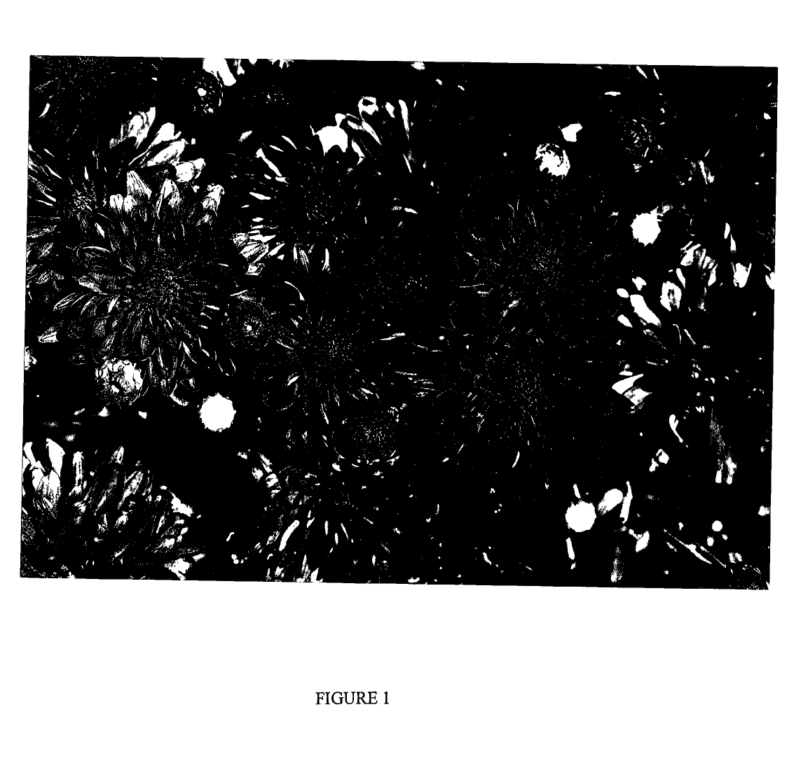

### BACKGROUND OF THE INVENTION

The present invention relates to a new and distinct cultivar of Chrysanthemum plant, botanically known as Dendranthema morifolium and herein after referred to by the cultivar name ‘GEDI ONE MIS.’

This new cultivar originated from hybridization of a female parent, cultivar ‘Marenco’ (unpatented in the United States), and a male parent cultivar ‘Bronia’ (unpatented in the United States). The new cultivar was discovered by the inventor in October 1996.

Asexual reproduction of the new cultivar by apical tip cutting and meristem tissue culture was performed in Oxnard, Calif. and has shown that the unique features of this new cultivar are stable and reproduced true to type on successive generations.

### SUMMARY OF THE INVENTION

The cultivar ‘GEDI ONE MIS’ has not been observed under all possible environmental conditions. The phenotype may vary somewhat with variations in environment such as temperature, daylength, and light intensity, without, however, any variance in genotype.

The following traits have been repeatedly observed and are determined to be the unique characteristics of ‘GEDI ONE MIS.’ These characteristics in combination distinguish ‘GEDI ONE MIS’ as a new and distinct Chrysanthemum: 1. Decorative type inflorescence; 2. Consistent flowering response to short days, blooming consistently after 49 days of short day length; 3. Free branching habit; and 4. Compact growth habit.

Plants of the new cultivar are similar to plants of the parent cultivar, ‘Marenco,’ in most horticultural characteristics, however plants of the new cultivar differed from plants of the cultivar ‘Marenco’ in the following characteristics: 1. The new cultivar naturally blooms 2 weeks earlier; 2. The new cultivar has blooms more uniformly; 3. The new cultivar has more blooms per flowering branch; and 4. The new cultivar has more ray florets.

Plants of the new cultivar are also similar to plants of the parent cultivar, ‘Bronia,’in most horticultural characteristics, however plants of the new cultivar differed from plants of the cultivar ‘Bronia’ in the following characteristics: 1. The new cultivar has different ray floret color; 2. The new cultivar has more blooms per flowering branch; and 3. The new cultivar has a wider diameter bloom.

The new cultivar can be compared to the Chrysanthemum cultivar ‘Jenny Wren’ (disclosed in Plant Patent No. 10,213). However plants of the new Chrysanthemum differed from the plants of the cultivar ‘Jenny Wren’ in the following characteristics: 1. The new cultivar has more ray florets; 2. The new cultivar has a more round plant shape and greater branching; 3. The new cultivar has a shorter internode length; 4. The new cultivar has a more flexible plant form; and 5. The new cultivar has more blooms per flowering branch.

### BRIEF DESCRIPTION OF THE PHOTOGRAPHS

The photograph shows a close up of the blooms, buds and leaves of ‘GEDI ONE MIS.’ Plants were grown under outdoor field conditions.

### DETAILED BOTANICAL DESCRIPTION

In the following description, color references are made to the Royal Horticultural Society Colour Chart except where general terms of ordinary dictionary significance are used. The following observations and measurements describe plants grown in a commercial filed on Oxnard, Calif. in the month of September 2000. The growing conditions range from 60° to 65° F. at night to 70 ° to 85° during the day. Measurements and numerical values represent averages of typical flowering types. The plant described was planted in mid-May and was grown in a twogallon container. The plant was pinched back 10 days after planting. Botanical classification: Dendranthema morifolium cultivar ‘GEDI ONE MIS’ Commercial classification: Garden-type Chrysanthemum Parentage: Hybridization of female parent ‘Marenco’ (unpatented in the United States) and male parent ‘Bronia’ (unpatented in the United States)

PROPAGATION

Time to Rooting.—7 to 14 days at approximately 21° C.

Rooting habit.—Fine, fibrous

PLANT

Growth Habit.—Mounding herbaceous perennial

Height.—Approximately 29 cm

Growth rate.—Moderate

Branching Characteristics.—Free Branching

Length of Lateral Branches.—Approximately 24 cm

Number of leaves per lateral branch.—Approximately 21

Stem color.—Near 137A

FOLIAGE

Leaf.—Arrangement: Alternate single Average Length: Approximately 6.2 cm Average Width: Approximately 3.7 cm Shape of blade: Ovate Apex: Acuminate Base: Attenuate Attachment: Stalked Margin: Serrate Texture of top surface: Lightly pubescent Texture ofbottom surface: Lightly pubescent Color youngfoliage upper side: Near 138A youngfoliage under side: Near 138A mature foliage upper side: Near 137A mature foliage underside: Near 138A venation upper side: Near 137C venation under side: Near 137C

Petiole.—Average Length: Approximately 2.2 cm

BLOOM

Inflorescence.—Flowering habit: Induced by darkness period greater than 13.5 hours, approximately 49 days of appropriate day length required to induce and develop blooms Inflorescenceform: Decorative Naturalflowering season: Approximately mid-September Number of inflorescences per stem: Approximately 11 Inflorescence diameter: Approximately 7.2 cm Inflorescence depth: Approximately 2.2 cm Inflorescence longevity on plant: Approximately 22-26 days Persistence: Persistent Ray Florets: Appearance: Matte Texture: Smooth Average Numberperflower: Approximately 300 Shape: Oblanceolate Margin: Entire Apex: Obtuse Length: Approximately 3.9 cm Width: Approximately 1.0 cm Color: upper surface at first opening: Near 178A upper surface at maturity: Near 180A upper surface at fading: Near 180B under surface at first opening: Near 180A under surface at maturity: Near 180A under surface at fading: Near 180C

Disc florets.—Appearance: Shiny Texture: Smooth Average number per flower: Approximately 60 Shape: Cylindric Apex: Obtuse Average Length: Approximately 1.0 cm Average Width: Approximately 0.2 cm Color: at first opening: Near 3D at maturity: Near 16A at fading. Near 16C

Peduncle.—Length: at terminal end (shortest): Approximately 5 cm at lateral end (longest): Approximately 8 cm Strength: Moderate Color: Near 138B Habit: Erect

Inflorescence bud.—Length: Approximately 1.0 cm Diameter: Approximately 1.0 cm Form: Globular Color: Near 178A

Sepals.—Appearance: Mafte Texture: Lightly pubescent Number: Approximately 30 to 40 Shape: Ovate Margin: Entire Apex: Acute Color upper side: Near 137B under side: Near 137B

REPRODUCTIVE ORGANS

Ray Florets.—Pistillate: Number of pistils per flower: 1 Stigma shape: 2 branched Stigma color: Near 10B Style color: Near 10D Style length: Approximately 0.9 cm

Disc Florets.—Pistillate: Number ofpistils per flower: 1 Stigma shape: Cylindric Style length: Approximately 0.5 cm Staminate: Number of stamens per flower: Approximately 5 Anther shape: Tubular Anther color: Near 18A Pollen color: Near 17C

OTHER CHARACTERISTICS

Disease Resistance.—Neither resistance nor susceptibility observed

What is claimed is:

1. A new and distinct variety of Chrysanthemum plant named ‘GEDI ONE MIS’ as herein illustrated and described.
